(19) United States
(12) Patent Application Publication
(10) Pub. No.: US 2012/0283498 A1
(43) Pub. Date: Nov. 8, 2012

(54) PROCESS FOR ISOMERIZATION OF A C8 AROMATIC FRACTION IN THE PRESENCE OF A SPECIFIC CATALYST THAT CONSISTS OF A ZEOLITE/SILICON CARBIDE-TYPE COMPOSITE AND A HYDROGENATING-DEHYDROGENATING FUNCTION

(75) Inventors: Emmanuelle Guillon, Vourles (FR); Christophe Bouchy, Lyon (FR); Eric Sanchez, Saint Genis Laval (FR)

(73) Assignee: IFP Energies nouvelles, Rueil-Malmaison Cedex (FR)

(21) Appl. No.: 13/463,403

(22) Filed: May 3, 2012

(30) Foreign Application Priority Data
May 4, 2011 (FR) 11/01.375

(51) Int. Cl. C07C 5/22

(52) U.S. Cl. 585/481

(57) ABSTRACT

A process for isomerization of an aromatic fraction that contains at least one aromatic compound that has eight carbon atoms per molecule is described, with said process comprising bringing said fraction into contact with a catalyst that comprises at least one hydrogenating-dehydrogenating metal that is selected from the group that is formed by the metals of group VIB and group VIII of the periodic table and a composite that is formed by a zeolite that is selected from among the EUO-, MTW- and MOR-structural type zeolites and silicon carbide SiC.

### FIELD OF THE INVENTION

This invention relates to the isomerization of an aromatic fraction that contains at least one aromatic compound with eight carbon atoms per molecule for the purpose of the production of xylenes, and, in particular, paraxylene. Said aromatic fraction that is used in the process according to this invention is a feedstock that contains a mixture of xylenes, ethylbenzene, or a mixture of xylenes and ethylbenzene. Said feedstock is commonly called “C8 aromatic fraction.”

This invention relates more particularly to a process for isomerization of an aromatic feedstock that comprises at least one aromatic compound with eight carbon atoms per molecule whose object is to maximize the production of paraxylene.

### PRIOR ART

The catalysis of the isomerization of ethylbenzene into xylenes calls for the presence of a metal from group VIII. The optimized formulations based on mordenite and a metal from group VIII lead to catalysts with which the parasitic reactions are non-negligible. For example, it is possible to cite the opening of naphthene rings, followed or not followed by cracking, or else reactions for dismutation and transalkylation of C8 aromatic compounds, which lead to the formation of undesirable aromatic compounds. It is therefore particularly advantageous for finding new, more selective and/or more active catalysts.

Among the zeolites that are used for isomerization of C8 aromatic fractions, there is ZSM-5, used by itself or in a mixture with other zeolites, such as, for example, mordenite. These catalysts are described in particular in the patents U.S. Pat. No. 4,467,129, U.S. Pat. No. 4,482,773 and EP-B-13 617. Other primarily mordenite-based catalysts have been described in, for example, the patents U.S. Pat. No. 4,723,051, U.S. Pat. No. 4,665,258 and FR-A-2 477 903. More recently, a catalyst based on an EUO-structural-type zeolite was proposed (EP-A1-923 987). The patent application WO-A-2005/065 380 describes the use of an MTW-structural-type zeolite for isomerization of xylenes and ethylbenzene.

The patent application FR 2 915 112 teaches the use of a process for isomerization of a “C8 aromatic” fraction that comprises bringing said fraction into contact with a catalyst that contains an EUO-structural-type zeolite, whereby said catalyst has been prepared according to a process that comprises at least the following stages: i) the synthesis of at least one EUO-structural-type zeolite that has an overall Si/Al atomic ratio of between 5 and 45; ii) the dealuminification of the zeolite that is obtained at the end of said stage i) in such a way that at least 10% by mass of the aluminum atoms are extracted from said zeolite that is obtained from said stage i); iii) the shaping of said dealuminified zeolite with a matrix; iv) the deposition of at least one metal from group VIII of the periodic table, where the sequence in which said stages iii) and iv) are carried out is unimportant following said stage ii).

The patent FR2834655 teaches the synthesis of zeolite/SiC composites and their use in catalysis and in particular of ZSM-5/SiC, β/SiC, and silicalite/SiC composites, with the preferred composite being the ZSM-5/SiC composite. This patent does not teach the potential use of a composite that is formed by an EUO-, MTW- or MOR-structural type zeolite and silicon carbide SiC for isomerization of a C8 aromatic fraction and no longer teaches the implementation of specific post-treatments of the composite so as to adjust the level of acidity and/or the porosity of the zeolite of said composite.

By attempting to develop active catalysts for the isomerization of a C8 aromatic fraction, the applicant discovered that the use of a catalyst that comprises at least one hydrogenating-dehydrogenating metal that is selected from the group that is formed by the metals of group VIII and a composite substrate that comprises a specific zeolite and silicon carbide SiC in a process for isomerization of a C8 aromatic fraction made it possible to obtain improved catalytic performance levels in terms of the activity of said catalyst.

### SUMMARY AND ADVANTAGE OF THE INVENTION

This invention relates to a process for isomerization of an aromatic fraction that contains at least one aromatic compound that has eight carbon atoms per molecule, whereby said process comprises bringing said fraction into contact with a catalyst that comprises at least one hydrogenating-dehydrogenating metal that is selected from the group that is formed by the metals of group VIII of the periodic table and a composite substrate that comprises silicon carbide SiC and a zeolite that is selected from among the EUO-, MTW- or MOR-structural-type zeolites, taken by itself or in a mixture.

It was thus discovered, surprisingly enough, that a catalyst that comprises a composite substrate that comprises silicon carbide and a zeolite that is selected from among the EUO-, MTW- and MOR-structural-type zeolites, taken by itself or in a mixture, leads to improved catalytic performance levels in terms of activity and in particular in terms of xylene yield when it is used in a process for isomerization of an aromatic fraction that comprises at least one aromatic compound with eight carbon atoms per molecule. In particular, such a catalyst is more active than a catalyst of the prior art that comprises a non-composite substrate that is based on an EUO-, MTW-, or MOR-structural-type zeolite by itself and that does not use a composite substrate that comprises silicon carbide and an EUO-, MTW-, or MOR-structural-type zeolite.

### DESCRIPTION OF THE INVENTION

This invention has as its object a process for isomerization of an aromatic fraction that contains at least one aromatic compound that has eight carbon atoms per molecule and that comprises bringing said fraction into contact with a catalyst that comprises at least one hydrogenating-dehydrogenating metal that is selected from the group that is formed by the metals of group VIII of the periodic table and a composite substrate that comprises silicon carbide SiC and at least one zeolite that is selected from among the EUO-, MTW- and MOR-structural-type zeolites, taken by itself or in a mixture.

Preferably, said composite substrate of the catalyst that is used in the process according to the invention comprises and is preferably composed of, in percentage by weight relative to the total mass of said substrate:
20 to 99.5%, in a preferred manner 50 to 99.5%, of silicon carbide (SiC), whose specific surface area that is measured by the BET method is greater than 5 m²/g;
0.5 to 80%, in a preferred manner from 0.5 to 50%, of a zeolite that is selected from among the EUO-, MTW- and MOR-structural-type zeolites, taken by itself or in a mixture.

### Silicon Carbide

According to the invention, the silicon carbide that is used in the substrate of the catalyst that is employed in the process according to the invention has a specific surface area, measured by the BET technique, that is greater than 5 m²/g, preferably between 5 and 300 m²/g, and in an even preferable manner between 10 and 250 m²/g.

The pore volume of the silicon carbide is advantageously between 0.20 cm³/g and 1.0 cm³/g, preferably between 0.25 cm³/g and 0.80 cm³/g, and in an even preferable manner between 0.25 cm³/g and 0.75 cm³/g.

The values of BET specific surface area were determined by nitrogen adsorption at the temperature of liquid nitrogen according to the BET method that is known to one skilled in the art.

The manufacturing of silicon carbide-type substrates that can be used in heterogeneous catalysis is already known and taught in patents such as in particular the patent EP 0 440 569 B1 or else the patent U.S. Pat. No. 6,184,178 B1, without this list being limiting. This type of substrate is industrially manufactured by, for example, the SICAT Sarl Company.

### The Zeolite

According to the invention, the zeolite used in the composite substrate of the catalyst that is employed in the process according to the invention is selected from among the EUO-, MTW- and MOR-structural-type zeolites, taken by themselves or in a mixture.

Preferably, the EUO-structural-type zeolite is selected from among the EU-1, TPZ-3 and ZSM-50 zeolites, taken by themselves or in a mixture, and in a preferred manner, the EUO-structural-type zeolite is the EU-1 zeolite.

Preferably, the MTW-structural-type zeolite is selected from among the ZSM-12, NU-13, and TPZ-12 zeolites, taken by themselves or in a mixture, and in a preferred manner, the MTW-structural-type zeolite is the ZSM-12 zeolite.

Preferably, the MOR-structural-type zeolite is mordenite.

In a preferred manner, the zeolite that is used in the composite substrate of the catalyst that is employed in the process according to the invention is the EUO-structural-type zeolite and, in a very preferred manner, the EU-1 zeolite.

The EUO-, MTW- and MOR-structural-type zeolites that are described are well known in the prior art, and their porous structure and topography are defined in “The Atlas of Zeolite Framework Types,” Ch. Baerlocher, W. M. Meier, D. H. Olson, 7^{th} Edition, 2007. The EUO-type zeolites have a network of one-dimensional medium-sized pores (10 MR) with side pockets with 12 tetrahedral atoms (12 MR). The MOR- and MTW-type zeolites have a one-dimensional network with large pores (12 MR).

The mode of preparation of the different types of zeolites, i.e., the composition of the liquid reaction media that are suitable for the preparation of different EUO-, MTW-, or MOR-structural-type zeolites, is well known to one skilled in the art and is explained below.

### Synthesis of the Zeolite/Silicon Carbide Composite Substrate

The composite substrate that comprises silicon carbide SiC and at least one zeolite that is selected from among the EUO-, MTW- and MOR-structural-type zeolites, taken by themselves or in a mixture, is obtained by germination and growth of zeolite crystals on the surface of the silicon carbide that is already preformed, i.e., already in any form that makes possible the subsequent use of the catalyst that is prepared starting from this composite in an industrial catalytic unit.

By way of example, the preformed silicon carbide can advantageously come in the form of balls, extrudates, and even foam. The silicon carbide can advantageously undergo a heat treatment, preferably a calcination, before the zeolitization stage.

The zeolitization of the silicon carbide can advantageously be carried out by any method that is known to one skilled in the art.

According to a preferred embodiment, the zeolitization of the silicon carbide is implemented according to the conventional hydrothermal method. The silicon carbide is then advantageously immersed in the liquid reaction medium that contains the sources of the elements of the zeolitic frame, the sources of mineralizing agent (OH⁻), mineral cations and/or organic radicals, and a solvent (in general, water). The reaction mixture and the solid are next introduced into an autoclave, and then brought to temperature for a period that is suitable for the germination and growth of zeolite crystals on the surface of the silicon carbide.

According to another preferred embodiment, the zeolitization of the silicon carbide is carried out by application of the method called “dry gel conversion.” A dry impregnation of the silicon carbide is then advantageously carried out by the liquid reaction medium, and then the solid advantageously undergoes a hydrothermal treatment in an autoclave.

The EUO-, MTW- and MOR-structural-type zeolites, used by themselves or in a mixture in the catalyst that is used for the implementation of the isomerization process according to the invention, are preferably the zeolites that are selected from among the EU-1 zeolite, the TPZ-3 zeolite, and the ZSM-50 zeolite, and in a preferred manner, the EU-1 zeolite for the EUO-structural-type zeolites, preferably the zeolites that are selected from among the ZSM-12 zeolite, the NU-13 zeolite, and the TPZ-12 zeolite, and in a preferred manner, the ZSM-12 zeolite for the MTW-structural-type zeolites, and preferably mordenite for the MOR-structural-type zeolite.

The mode of preparation of the different types of zeolites, i.e., the composition of the liquid reaction media that are suitable for the preparation of the different EUO-, MTW- or MOR-structural type zeolites, is well known to one skilled in the art. In a general manner, the methods for preparation of such zeolites comprise the mixing in an aqueous medium of a silicon source, an aluminum source, a source of an alkaline metal, and a nitrogen-containing organic compound that plays the role of structuring agent.

The EU-1 zeolite, described in the European patent application EP-A-0 042 226, is prepared by using as structuring agent either the alkyl-containing derivative of a polymethylene α-ω diammonium or a degradation product of said derivative or else precursors of said derivative. The TPZ-3 zeolite, described in the European patent application EP-A-0 051 318, is prepared by using the same family of structuring agent as the one used for synthesizing the EU-1 zeolite. The use of the compound 1,6-N,N,N,N′,N′,N′-hexamethylhexamethylenediammonium is described in particular. The ZSM-50 zeolite, described in the documents EP 0 159 845 and U.S. Pat. No. 4,640,829, is prepared by using the dibenzyldimethylammonium (DBDMA) derivative as a structuring agent.

The synthesis of the ZSM-12 zeolite is described in the articles by Lapierre et al. (Zeolites, 5, 346 (1985)) and Fyfe et al. (J. Phys. Chem 94, 3718 (1990)).

Without being exhaustive, the ZSM-12 zeolite in aluminosilicate form can be synthesized by using a methyltriethylammonium chloride MTEACI-type organic structuring agent, methyltriethylammonium bromide TEABr as described in the patent application U.S. Pat. No. 6,893,624.

The synthesis of mordenite is described, in a non-exhaustive manner, by Meier et al. (Kristallogr 115 (1961)). Kim et al. (Zeolites 11 (1991) 745) describe the synthesis of the mordenite zeolite in the absence of a nitrogen-containing organic structuring agent by employing a mixture of soda, sodium aluminate, and silica.

Also, for the composition of the liquid reaction mixture to be used for the zeolitization of silicon carbide, one skilled in the art will usefully refer to one or the other of the references cited above.

For example, for the preparation of an EU-1 zeolite/silicon carbide composite substrate according to the conventional hydrothermal method, the preformed silicon carbide is immersed, for example, in the form of balls or extrudates in an aqueous medium that contains at least one silicon source, at least one aluminum source, and at least one nitrogen-containing organic structuring agent Q of formula R₁R₂R₃—N⁺—(CH₂)ₙ—N⁺—R₄R₅R₆, in which n is between 3 and 12, and the groups R₁ to R₆, identical or different, are alkyl groups that have 1 to 8 carbon atoms, with up to five of said groups R₁ to R₆ able to be hydrogen. The reaction mixture has the molar composition that is defined by the different typical molar ratios below:

| | |
|---|---|
| SiO₂/Al₂O₃ | 5-200 |
| OH⁻/SiO₂ | 0.1-6 |
| (M⁺ + Q)/Al₂O₃ | 0.5-200 |
| Q/(M⁺ + Q) | 0.1-10 |
| H₂O/SiO₂ | 1-100, |

with Q being the cation R₁R₂R₃—N⁺—(CH₂)ₙ—N⁺—R₄R₅R₆, described above, preferably 1,6 N,N,N,N′,N′,N′-hexamethylhexamethylene diammonium, and M⁺ being an alkaline cation or ammonium.

After zeolitization, the material is next filtered (in the case where the conventional hydrothermal method is implemented), washed with copious amounts of distilled water, dried, and optionally calcined. It can next undergo an ultrasonication stage so as to eliminate the zeolite crystals that are inadequately attached to the surface of the silicon carbide. The thus obtained composite optionally can be zeolitized again one or more times.

After drying and optional calcination, different characterizations, such as, for example, nitrogen porosimetry, X-ray diffraction, scanning electronic microscopy or else NMR for rotation at the magic angle of aluminum 27 can be carried out on the composite in such a way as to demonstrate the presence of zeolite crystallites. One of the advantages of the invention is to obtain zeolite crystals of smaller size than by the techniques that are known by one skilled in the art. Preferably, the zeolite crystals of the composite are of a size that is less than 2 microns, in a preferred manner less than 1 micron, and in a very preferred manner less than 0.5 micron.

The composite is next advantageously replaced by at least one treatment by a solution of at least one ammonium salt, such as, for example, ammonium nitrate or ammonium chloride, ammonium acetate, in such a way as to eliminate at least in part, preferably virtually totally, the alkaline cation, in particular sodium, optionally present in cationic position in the zeolite in its crude synthesis form. After replacement, the Na/Al atomic ratio of the zeolite is generally advantageously less than 0.1 and preferably less than 0.05 and in an even more preferred manner less than 0.01. The exchange stage can be carried out before or after the deposition of the hydrogenating-dehydrogenating phase, advantageously before.

The composite can also advantageously undergo a dealuminification stage so as to obtain the desired Si/Al ratio. This dealuminification stage can be carried out by any method that is known to one skilled in the art, such as, for example, a hydrothermal treatment, a dealuminification by chemical means, or else a combination of two methods (“Hydrocracking Science and Technology” of J. Scherzer and A. J. Gruia, Marcel Dekker, Inc., 1996). This dealuminification stage can advantageously be carried out before or after the stages for exchange and deposition of the hydrogenating-dehydrogenating phase, preferably after the exchange stage and before the stage for deposition of the hydrogenating-dehydrogenating phase.

According to a first embodiment of the dealuminification stage, the zeolite/silicon carbide composite is subjected to a calcination in a dry air stream, at a temperature of between 400 and 600° C., and then it is subjected to at least one treatment by an aqueous solution of a mineral acid or an organic acid. The calcination period is variable and is advantageously between several hours and several days. The treatment by calcination of the zeolite/silicon carbide composite has as its object to eliminate the organic structuring agent that is present in the microporosity of the zeolite, for example the cation R₁R₂R₃—N⁺—(CH₂)ₙ—N⁺—R₄R₅R₆, defined above, and preferably 1,6 N,N,N,N′,N′,N′-hexamethylhexamethylene diammonium when the zeolite of the composite is the EU-1 zeolite. The percentage by mass of residual carbon of the zeolite at the end of said calcination stage is preferably less than 0.3% and in a more preferred manner less than 0.1% relative to the zeolite mass.

Said treatment of the zeolite/silicon carbide composite by an aqueous solution of a mineral or organic acid that is carried out following the calcination stage is also called “acid attack stage.” Said treatment can be repeated as many times as it is necessary so as to obtain the desired level of dealuminification. In this case, the composite is washed with distilled water between each successive acid attack stage.

So that the zeolite of the composite has the overall Si/Al atomic ratio that is desired, it is necessary to select and monitor the operating conditions of each acid attack stage properly. In particular, the temperature at which the treatment by the aqueous solution of the mineral or organic acid is carried out, the nature and the concentration of the acid that is used, the ratio between the quantity of acid solution and the mass of treated composite, the period of the treatment, and the number of treatments carried out are significant parameters for the implementation of each acid attack stage. Advantageously, the treatment of the composite by an aqueous solution of a mineral acid or an organic acid is carried out at a temperature of between 30° C. and 120° C., preferably between 50° C. and 120° C., and in a very preferred manner between 60 and 100° C. The concentration of the acid in the aqueous solution is advantageously between 0.05 and 20 mol.L⁻¹, preferably between 0.1 and 10 mol.L⁻¹, and in an even more preferred manner between 0.5 and 5 mol.L⁻¹ The ratio between the volume V of acid solution in ml and the mass M of treated composite in g is advantageously between 1 and 50, preferably between 2 and 20. The period of the acid attack is advantageously greater than 1 hour, preferably between 2 hours and 10 hours, and in a preferred manner between 2 hours and 8 hours. The acid that is selected for the implementation of said acid attack stage is advantageously either a mineral acid or an organic acid; preferably, it is a mineral acid that is selected from among nitric acid HNO₃, hydrochloric acid HCl, and sulfuric acid H₂SO₄. In a very preferred manner, it is nitric acid. When an organic acid is used for the acid attack, the acetic acid CH₃CO₂H is preferred. The number of successive treatments of the composite by an acid aqueous solution is preferably less than 4. In the case where several successive acid attacks are carried out, aqueous solutions of mineral or organic acid of different acid concentrations advantageously can be used.

After having carried out treatment(s) by the acid aqueous solution, the composite is next advantageously washed with distilled water and then is dried at a temperature of between 80 and 140° C. for a period of between 10 and 48 hours.

According to a second embodiment of the dealuminification stage, the zeolite/silicon carbide composite is subjected to a calcination in a dry air stream, at a temperature of between 400 and 600° C., and then with one or more ion exchange(s) by at least one NH₄NO₃ solution, and then it is subjected to at least one cycle for dealuminification of the zeolitic framework that comprises at least one heat treatment that is carried out in the presence of water vapor and at least one acid attack by at least one aqueous solution of a mineral or organic acid.

The period of the calcination is variable and is advantageously encompassed between several hours and several days. The treatment by calcination of the zeolite/silicon carbide composite has as its object to eliminate the organic structuring agent that is present in the zeolite microporosity, for example the cation R₁R₂R₃—N⁺—(CH₂)ₙ—N⁺—R₄R₅R₆ that is defined above, and preferably 1,6 N,N,N,N′,N′,N′-hexamethylhexamethylene diammonium when the zeolite of the composite is the EU-1 zeolite. The percentage by mass of residual carbon of the zeolite at the end of said calcination stage is preferably less than 0.3% and in a more preferred manner less than 0.1% relative to the zeolite mass. The ion exchange(s) subsequent to said calcination in a dry air stream make(s) it possible to eliminate at least partly, preferably virtually totally, the alkaline cation, in particular sodium, optionally present in cationic position in the zeolite in its crude synthesis form. Each exchange is carried out at a temperature that is preferably between 50 and 150° C. for a period that is advantageously between 2 hours and 10 hours. In general, an aqueous solution of ammonium nitrate NH₄NO₃ that has a normality of between 0.5 and 12 N is used.

The operating conditions of the heat treatment in the presence of water vapor—in particular the temperature and the period of said treatment as well as the volumetric percentage of water vapor, as well as the operating conditions of the post-heat-treatment acid attack, in particular the acid attack period, the nature and the concentration of the acid that is used, and the ratio between the volume of acid solution and the mass of treated composite—are adapted in such a way as to obtain an EUO, MTW or MOR zeolite of the desired overall Si/Al atomic ratio. Advantageously, the heat treatment in the presence of water vapor is carried out at a temperature of between 200 and 900° C., preferably between 300 and 900° C., and in an even more preferred manner between 400 and 600° C. The period of said heat treatment is advantageously greater than or equal to 0.5 hour, preferably between 0.5 hour and 24 hours, and very preferably between 0.5 hour and 12 hours. The volumetric percentage of water vapor during the heat treatment is advantageously between 5 and 100%, preferably between 20 and 100%, and in an even more preferred manner between 40% and 100%. The volumetric fraction other than the water vapor that is optionally present is formed by air. The flow rate of gas formed by water vapor and optionally air is advantageously between 0.2 l/h/g of treated composite and 10 l/h/g of treated composite.

The temperature at which the acid attack is carried out, subsequent to the heat treatment in the presence of water vapor, is advantageously between 30° C. and 120° C., preferably between 50 and 120° C., and in a very preferred manner between 60 and 100° C. The concentration of acid in the aqueous solution is advantageously between 0.05 and 20 mol.L⁻¹, preferably between 0.1 and 10 mol.L⁻¹, and in an even more preferred manner between 0.5 and 5 mol.L⁻¹. The ratio between the volume of acid aqueous solution V in ml and the treated composite mass M in g is advantageously between 1 and 50, preferably between 2 and 20. The period of the acid attack is advantageously greater than 1 hour, preferably between 2 hours and 10 hours, and in a preferred manner between 2 hours and 8 hours. The acid that is selected for the implementation of the acid attack is either a mineral acid or an organic acid; preferably it is a mineral acid that is selected from among nitric acid HNO₃, hydrochloric acid HCl, and sulfuric acid H₂SO₄. In a very preferred manner, it is nitric acid. When an organic acid is used for the acid attack, the acetic acid CH₃CO₂H is preferred.

The dealuminification cycle of the zeolitic framework that comprises at least one heat treatment carried out in the presence of water vapor and at least one acid attack by at least one aqueous solution of a mineral or organic acid can be repeated as many times as it is necessary to obtain the dealuminified zeolite, having the desired Si/Al atomic ratio. The dealuminification cycle number is preferably less than 4.

According to a third embodiment of the dealuminification stage, the EUO-, MTW- or MOR-structural-type zeolite/silicon carbide composite is subjected to a heat treatment that is carried out in the presence of water vapor at a temperature of between 450 and 850° C., and then with at least one treatment by an aqueous solution of a mineral or organic acid. Said treatment(s) by an acid aqueous solution is (are) preferably followed by one or more ion exchange(s) by at least one NH₄NO₃ solution in such a way as to eliminate virtually the entire alkaline cation, in particular sodium, optionally present in cationic position in the zeolite in its crude synthesis form. Each exchange is carried out at a temperature that is preferably between 50 and 150° C. for a period that is advantageously between 2 hours and 10 hours. In general, an aqueous solution of ammonium nitrate NH₄NO₃ that has a normality of between 0.5 and 12 N is used.

In accordance with said third embodiment of the dealuminification stage, said heat treatment in the presence of water vapor is carried out simultaneously with the elimination of the organic structuring agent that is present in the microporosity of said zeolite, for example the cation R₁R₂R₃—N⁺—(CH₂)ₙ—N⁺—R₄R₅R₆, defined above, and preferably 1,6 N,N,N,N′,N′,N′-hexamethylhexamethylene diammonium when the zeolite of the composite is the EU-1 zeolite. Actually, the heat treatment in the presence of water vapor is carried out at a temperature that is high enough to allow the elimination of said structuring agent. The operating conditions for implementing said heat treatment in the presence of water vapor are identical to those that are provided above for carrying out the heat treatment in the presence of water vapor implemented in the dealuminification cycle of the second embodiment of the dealuminification stage. The treatment by an aqueous solution of a mineral or organic acid is carried out under the same operating conditions as those provided above for carrying out the acid attack by at least one aqueous solution of a mineral or organic acid that is implemented in the dealuminification cycle of the second embodiment of the dealuminification stage. Following the heat treatment that is carried out in the presence of water vapor, several successive acid attacks can advantageously be performed in such a way as to obtain the desired level of dealuminification. The acid aqueous solutions used for implementing these different acid attack stages have an identical or different concentration, preferably different. Between each acid attack, the compound that is obtained is washed with distilled water.

### Hydrogenating-Dehydrogenating Function.

The catalyst that is used in the process according to the invention comprises at least one hydrogenating-dehydrogenating metal that is selected from the group that is formed by the metals of group VIII of the periodic table. Preferably, said metal of group VIII is selected from among iron, cobalt, nickel, ruthenium, rhodium, palladium, osmium, iridium and platinum, taken by itself or in a mixture. In a preferred manner, said metal of group VIII is selected from among the noble metals, taken by itself or in a mixture, and in a very preferred manner from among palladium and platinum, taken by itself or in a mixture. In an even more preferred manner, said metal of group VIII is platinum.

Said catalyst also optionally comprises at least one metal that is selected from among the metals of groups IIIA, IVA, and VIIB, taken by itself or in a mixture.

Said metals of groups IIIA, IVA and VIIB are advantageously selected from among gallium, indium, tin, and rhenium, taken by itself or in a mixture, and preferably from among indium, tin and rhenium, taken by itself or in a mixture.

### Deposition of the Hydrogenating-Dehydrogenating Function

The preparation of the catalyst that is used in the isomerization process according to the invention can be carried out by any method that is known to one skilled in the art. The hydrogenating-dehydrogenating function advantageously can be introduced at any stage of the preparation, in a very preferred manner after the stages of zeolitization, exchange and dealuminification if the latter is performed. The deposition of the hydrogenating-dehydrogenating function is advantageously performed by the dry impregnation technique, the technique of impregnation by excess or by ion exchange. When several metals are introduced, the latter can be introduced either all in the same way or by different techniques.

All of the precursors of the metals of group VIII are suitable for the deposition of one or more metal(s) from group VIII on the composite. In particular, in the case where the metal of group VIII is a noble metal, it is possible to use ammoniated compounds or compounds such as, for example, ammonium chloroplatinate, dicarbonyl platinum dichloride, hexahydroxyplatinic acid, palladium chloride, or palladium nitrate. The platinum can also advantageously be introduced in the form of hexachloroplatinic acid. The introduction of the noble metal of group VIII is preferably carried out by impregnation using an aqueous or organic solution of one of the metal compounds cited above. Among the organic solvents that can be used, it is possible to cite the paraffinic, naphthenic or aromatic hydrocarbons that contain, for example, 6 to 12 carbon atoms per molecule, and the halogenated organic compounds that contain, for example, 1 to 12 carbon atoms per molecule. It is possible to cite, for example, n-heptane, methylcyclohexane, toluene and chloroform. It is also possible to use mixtures of solvents.

Thus, to introduce the metal(s) of group VIII, preferably platinum and/or palladium, it is possible to implement an anion exchange with hexachloroplatinic acid and/or hexachloropalladic acid, in the presence of a competing agent, for example, hydrochloric acid, with the deposition in general being followed by a calcination, for example at a temperature of between 350 and 550° C., and for a period of between 1 and 4 hours.

It is also possible to consider depositing the metal(s) of group VIII, preferably platinum and/or palladium, by cation exchange. Thus, in the case of platinum, the precursor can be selected from among, for example:
The ammoniated compounds such as the tetramine platinum (II) salts of formula Pt(NH₃)₄X₂; the hexamine platinum (IV) salts of formula Pt(NH₃)₆X₄; the halopentamine platinum (IV) salts of formula (PtX(NH₃)₅)X₃; the N-tetrahalodiamine platinum salts of formula PtX₄(NH₃)₂; and
The halogenated compounds of formula H(Pt(acac)₂X); X being a halogen that is selected from the group that is formed by chlorine, fluorine, bromine and iodine, X preferably being chlorine, and “acac” representing the acetylacetonate group (of empirical formula C₅H₇O₂), derived from acetylacetone.

In the case where the catalyst that is used in the isomerization process according to the invention also contains at least one metal that is selected from among the metals of groups IIIA, IVA and VIIB, all of the techniques for deposition of such a metal that are known to one skilled in the art and all of the precursors of such metals can be suitable.

It is possible to add the metal(s) of group VIII and that (those) of groups IIIA, IVA and VIIB either separately or simultaneously in at least one unit stage. When at least one metal of groups IIIA, IVA and VIIB is added separately, it is preferable that it be added after the metal of group VIII.

The additional metal that is selected from among the metals of groups IIIA, IVA and VIIB can be introduced by means of compounds such as, for example, chlorides, bromides, and nitrates of the metals of groups IIIA, IVA and VIIB. For example, in the case of indium, nitrate or chloride is advantageously used, and in the case of rhenium, perrhenic acid is advantageously used. In the case of tin, the tin chlorides SnCl₂ and SnCl₄ are preferred. The additional metal that is selected from among the metals of groups IIIA, IVA and VIIB can also be introduced in the form of at least one organic compound that is selected from the group that consists of the complexes of said metal, in particular the polyketonic complexes of the metal and the hydrocarbyl metals such as alkyl, cycloalkyl, aryl, alkylaryl and arylalkyl metals. In this latter case, the introduction of the metal is advantageously performed using a solution of the organometallic compound of said metal in an organic solvent. It is also possible to use organohalogenated compounds of the metal. As organic compounds of metals, it is possible to cite in particular tetrabutyltin, in the case of tin, and triphenylindium, in the case of indium.

If the additional metal that is selected from among the metals of groups IIIA, IVA and VIIB is introduced before the metal of group VIII, the compound of the metal IIIA, IVA and/or VIIB that is used is selected in general from the group that consists of halide, nitrate, acetate, tartrate, carbonate and oxalate of metal. The introduction is then advantageously performed in aqueous solution. It may also be introduced, however, using a solution of an organometallic compound of the metal, for example tetrabutyltin. In this case, before initiating the introduction of at least one metal of group VIII, a calcination in air will be initiated.

In addition, intermediate treatments, such as, for example, a calcination and/or a reduction, can be applied between the successive depositions of different metals.

In general, the preparation of the catalyst ends by a calcination, usually at a temperature of between 250° C. and 600° C., for a period of between 0.5 and 10 hours, preferably preceded by drying, for example in an oven, at a temperature that ranges from ambient temperature to 250° C., preferably from 40° C. to 200° C. Said drying stage is preferably conducted during the rise in temperature that is necessary for carrying out said calcination. It is possible to implement a preliminary reduction of the catalyst ex situ, under a hydrogen flow, for example at a temperature of 450° C. to 600° C., for a period of 0.5 to 4 hours.

The deposition of said metal(s) of group VIII is advantageously performed in such a way that the dispersion of said metal(s), determined by chemisorption, is 20% to 100%, preferably 30% to 100%, and in an even more preferred manner, 40% to 100%. The deposition of said metal(s) of group VIII is also advantageously performed in such a way as to obtain a good distribution of said metal(s) in the shaped catalyst. This distribution is characterized by its profile that is obtained by Castaing microprobe. The ratio of the concentrations of each element of group VIII at the core of the grain relative to the edge of this same grain, defined as being the distribution coefficient, is advantageously 0.7:1 to 1.3:1, preferably 0.8:1 to 1.2:1.

The catalyst that is used in the process according to the invention advantageously comprises a content of metal(s) of group VIII, preferably platinum, of between 0.01 and 4%, preferably between 0.05 and 2.0%, by weight relative to the total mass of the catalyst. The EUO-, MTW-, MOR-type zeolite/silicon carbide composite constitutes the make-up to 100%. When said catalyst contains at least one metal that is selected from among the metals of groups IIIA, IVA and VIIB, the content of the latter can range up to 2% by weight relative to the total mass of the catalyst. The content of metals of groups IIIA, IVA and VIIB is then advantageously between 0.01 and 2%, preferably between 0.05 and 1.0% by weight relative to the total mass of the catalyst.

When said catalyst contains sulfur, the content of the latter can be such that the ratio of the number of sulfur atoms to the number of metal atoms of group VIII that are deposited goes up to 2:1. It is then advantageously from 0.5:1 to 2:1. The EUO-, MTW-, or MOR-structural type zeolite that is present in the catalyst that is used in the isomerization process according to the invention preferably comes in its protonated form (hydrogen H⁺ form) which the proportion of cation other than H⁺ is less than 30% of the total number of cations, preferably less than 20%, and in a very preferred manner less than 10%, and in an even more preferred manner less than 5% relative to the total number of cations on the zeolite.

In the case where the catalyst does not contain sulfur, a reduction of the metal under hydrogen is advantageously carried out in situ before injection of the feedstock into the reaction medium.

In the case where the catalyst that is used in the invention contains sulfur, the sulfur is introduced onto the shaped catalyst, calcined, containing the metal or metals cited above, either in situ before the catalytic reaction, or ex situ. The optional sulfurization takes place after the reduction. In the case of an in situ sulfurization, the reduction, if the catalyst has not been reduced in advance, takes place before the sulfurization. In the case of an ex situ sulfurization, the reduction is carried out, and then the sulfurization. The sulfurization is performed in the presence of hydrogen by using any sulfurizing agent that is well known to one skilled in the art, such as, for example, dimethyl sulfide or hydrogen sulfide. For example, the catalyst is treated with a feedstock that contains dimethyl sulfide in the presence of hydrogen, with a concentration such that the sulfur/metal atomic ratio is 1.5. The catalyst is next kept for approximately 3 hours at approximately 400° C. under a stream of hydrogen before the injection of the feedstock.

### The Isomerization Process

The isomerization process according to the invention consists in bringing an aromatic fraction that contains at least one aromatic compound that has eight carbon atoms per molecule into contact with at least one catalyst that comprises at least one hydrogenating-dehydrogenating metal that is selected from the group that is formed by the metals of group VIII of the periodic table and a composite substrate that comprises a zeolite that is selected from among the EUO-, MTW-, and MOR-structural type zeolites, taken by itself or in a mixture, and silicon carbide SiC. The object of the isomerization process according to the invention is in particular the production of xylenes and preferably paraxylene.

Said aromatic fraction that contains at least one aromatic compound that has eight carbon atoms per molecule comprises in particular—as an aromatic compound that has eight carbon atoms per molecule—either a mixture of only xylenes, or only ethylbenzene, or a mixture of xylene(s) and ethylbenzene. It is commonly called “C8 aromatic fraction.”

Said isomerization process advantageously is performed at a temperature of between 300° C. and 500° C., preferably between 320° C. and 450° C., and even more preferably between 340° C. and 430° C.; at a partial hydrogen pressure of between 0.3 and 1.5 MPa, preferably between 0.4 and 1.2 MPa, and in an even preferable manner between 0.7 and 1.2 MPa; at a total pressure of between 0.45 and 1.9 MPa, preferably between 0.6 and 1.5 MPa; and at a feed volumetric flow rate, expressed in terms of kilogram of feedstock introduced per kilogram of catalyst and per hour, of between 0.25 and 30 h⁻¹, preferably between 1 and 10 h⁻¹, and in an even preferable manner between 2 and 6 h⁻¹.

The following examples illustrate the invention without thereby limiting its scope.

### Example 1

Preparation of an Isomerization Catalyst A in Accordance with the Invention

### a) Preparation of the EU-1-Type Zeolite/Silicon Carbide Composite

### Silicon Carbide

Silicon carbide is provided by the SICAT Company, in the form of cylindrical extrudates with a 1 mm diameter. Its primary characteristics are provided in Table 1 below.

TABLE 1
Primary Characteristics of the Silicon Carbide that is Used.

| Crystallized Phase (XRD) | β-SiC | |
|---|---|---|
| BET Specific Surface Area (N₂ | 25 | m²/g |
| Porosimetry) | | |
| Micropore Volume (N₂ Porosimetry) | Zero | |
| Median Pore Diameter (Hg Porosimetry) | 40 | nm |
| Total Pore Volume (Hg Porosimetry) | 0.35 | cm³/g |
| Shell-Type Bed Crushing | >3 | MPa |
| (Shell SMS 1471-74 Standard) | | |

| | |
|---|---|
| Impurities | Fe < 0.05% by Weight, |
| | Al < 0.1% by Weight, |
| | Ca < 0.01% by Weight |

### Synthesis of the EU-1-Type Zeolite/SiC Composite

50 grams of silicon carbide is calcined in air in a muffle furnace in a thin layer at 900° C. for 2 hours. The zeolitization of the substrate is next performed by hydrothermal treatment. The silicon carbide is immersed in a stainless steel Teflon autoclave in a liquid reaction mixture that has the following molar composition: 60 SiO₂:10.6 Na₂O:5.27 NaBr:1.5 Al₂O₃:19.5 Hexa-Br₂:2,777 H₂O, with Hexa-BR₂ being 1,6 N,N,N,N′,N′,N′-hexamethylhexamethylene diammonium, and bromine being the counter-ion. The quantity of reaction mixture is selected in such a way as to obtain an SiO₂/SiC mass ratio of 0.3. The Teflon autoclave is next stirred continuously (300 rpm) for 5 days at 180° C. The thus obtained mixture consists of EU-1 zeolite bulk crystals and EU-1/SiC composite extrudates that are easily recovered by sieving. The extrudates are next rinsed with distilled water until obtaining a pH of the washing water that is less than 9, and then undergo an ultrasonication stage for 30 minutes so as to eliminate the EU-1 crystals that are attached poorly or not at all to the silicon carbide. The extrudates are finally dried in an oven at 110° C. for 24 hours. The composite next undergoes a so-called dry calcination at 550° C. in a stream of dry air for 24 hours in such a way as to eliminate the organic structuring agent. Then, the solid that is obtained is subjected to four ion exchanges in a 10N solution of NH₄NO₃, at approximately 100° C. for 4 hours for each exchange. The quantity of zeolite in the composite was evaluated by its solubilization in an aqueous solution of hydrofluoric acid at 48% by weight for one hour at ambient temperature and by measurement of the resulting loss of weight of the solid. With this method, the zeolite content of the composite is evaluated at 10% by weight. The contents of aluminum and sodium in the composite that are measured by the mass spectrometry technique by induced plasma (ICP-MS) after mineralization of the sample correspond to an overall Si/Al atomic ratio of 16 and an Na/Al atomic ratio of 0.05. The presence of zeolite in the composite is also confirmed by the results of nitrogen porosimetry since the initial solid passes from a BET specific surface area of 25 m²/g and a zero micropore volume with a BET specific surface area of 75 m²/g and a micropore volume of 0.02 CM³/g.

### b) Deposition of the Hydrogenating-Dehydrogenating Function

The EU-1/SiC composite is subjected to an anion exchange with hexachloroplatinic acid in the presence of hydrochloric acid as a competing agent, in such a way as to deposit 0.2% by weight of platinum relative to the total mass of the catalyst. The moist solid is next dried at 120° C. for 12 hours and calcined in a flow of dry air at the temperature of 500° C. for one hour. The thus obtained catalyst is the catalyst A.

### Example 2 (Anomalous)

Preparation of an Isomerization Catalyst B with an EU-1 Zeolite Base that is Not in Accordance with the Invention.

### a) Preparation and Shaping of the EU-1-Type Zeolite

### Synthesis of the EU-1-Type Zeolite

This zeolite was synthesized in accordance with the teaching of the patent EP-B 1-0,042,226. In a stainless steel Teflon autoclave, a liquid reaction mixture that has the following molar composition was prepared: 60 SiO₂: 10.6 Na₂O:5.27 NaBr:1.5 Al₂O₃: 19.5 Hexa-Br₂: 2,777 H₂O, with Hexa-Br₂ being the 1,6 N,N,N,N′,N′,N′-hexamethylhexamethylene diammonium, and bromine being the counter-ion. The Teflon autoclave is next stirred continuously (300 rpm) for 5 days at 180° C.

This EU-1 zeolite first undergoes a so-called dry calcination at 550° C. in a stream of dry air for 24 hours in such a way as to eliminate the organic structuring agent. Then, the solid that is obtained is subjected to four ion exchanges in a 10N solution of NH₄NO₃ at approximately 100° C. for 4 hours for each exchange. The thus obtained solid has an overall Si/A1 atomic ratio of 15 and an Na/Al atomic ratio of 0.06.

Shaping of the EU-1-Type Zeolite with an Alumina Binder

The EU-1 zeolite that is obtained is next shaped by extrusion with an alumina gel in such a way as to obtain, after drying at a temperature that is equal to 100° C. for 1 night and a calcination in dry air that is conducted at a temperature that is equal to 450° C. for 4 hours, a solid in cylindrical extrudate form with a 1 mm diameter and containing 10% by weight of EU-1 zeolite and 90% alumina. The final solid has a specific surface area of 249 m²/g and a micropore volume of 0.015 cm³/g.

### b) Deposition of the Hydrogenating-Dehydrogenating Function

The EU-1/alumina cylindrical extrudates are next subjected to an anion exchange with hexachloroplatinic acid in the presence of hydrochloric acid as a competing agent in such a way as to deposit 0.2% by weight of platinum relative to the weight of the catalyst. The moist solid is next dried at 120° C. for 12 hours and calcined in a stream of dry air at the temperature of 500° C. for one hour. The thus obtained solid is the catalyst B.

### Example 3

### Evaluation of the Catalytic Properties of Catalysts A and B for Isomerization of Ethylbenzene.

The feedstock to be isomerized, brought into contact with the catalysts A and B, consists only of ethylbenzene.

The operating conditions of the isomerization are as follows:
Temperature: 390° C.;
Total pressure: 8 bar (1 bar=0.1 MPa);
Partial Hydrogen Pressure: 7 bar.
Feedstock: Ethylbenzene
Feed volumetric flow rate, expressed in terms of kilogram of feedstock introduced per kilogram of catalyst and per hour, equal to 5 h⁻¹.

The catalytic properties of the catalysts A and B for the isomerization of ethylbenzene are evaluated successively. Each of the catalysts is reduced under hydrogen for 4 hours at 480° C. before injection of the feedstock.

The catalysts have been evaluated in terms of the conversion of ethylbenzene and xylene selectivity.

The xylene selectivity is calculated by means of the yield of xylenes that are produced. The xylene yield is determined starting from the % by mass of the xylenes that are produced, obtained by analysis of each effluent.

The conversion of the ethylbenzene is the percentage of ethylbenzene that is consumed.

TABLE 1
Conversion of Ethylbenzene and Selectivity and Yield of Xylenes
on Catalysts A and B after 4000 Minutes of Reaction.

| | Catalyst A | Catalyst B |
|---|---|---|
| Ethylbenzene Conversion (%) | 37.4 | 25.2 |
| Xylene Selectivity (%) | 66.1 | 66.1 |
| Xylene Yield (%) | 24.7 | 16.7 |

The results presented in Table 1 show that the catalyst A that comprises an EU-1 zeolite/SiC composite substrate leads to much better catalytic performance levels in terms of ethylbenzene conversion than the one that is obtained by means of the catalyst B based on EU-1 zeolite alone.

Furthermore, the catalyst A according to the invention leads to a xylene selectivity that is identical to the one that is obtained with the catalyst B; consequently, the catalyst A according to the invention leads to a xylene yield that is much higher than the xylene yield that is obtained with the comparison catalyst B, with the xylene yield being the product of the conversion of ethylbenzene by the xylene selectivity.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding French application No. 11/01.375, filed 4 May 2011, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Process for isomerization of an aromatic fraction that contains at least one aromatic compound that has eight carbon atoms per molecule, whereby said process comprises bringing said fraction into contact with a catalyst that comprises at least one hydrogenating-dehydrogenating metal that is selected from the group that is formed by the metals of group VIII of the periodic table, and a composite substrate that comprises silicon carbide SiC and a zeolite that is selected from among the EUO-, MTW-, and MOR-structural type zeolites, taken by itself or in a mixture.

2. Process according to claim 1, in which said composite substrate consists of, in percentage by weight relative to the total mass of said substrate:
50 to 99.5% of silicon carbide (SiC), whose specific surface area that is measured by the BET method is greater than 5 m²/g;
0.5 to 50% of a zeolite that is selected from among the EUO-, MTW- and MOR-structural-type zeolites, taken by itself or in a mixture.

3. Process according to claim 1, in which the zeolite is the EUO-structural-type zeolite that is selected from among the EU-1, TPZ-3 and ZSM-50 zeolites, taken by themselves or in a mixture.

4. Process according to claim 3, in which the zeolite is the EU-1 zeolite.

5. Process according to claim 1, in which said metal of group VIII is selected from among iron, cobalt, nickel, ruthenium, rhodium, palladium, osmium, iridium and platinum, taken by itself or in a mixture.

6. Process according to claim 5, in which said metal of group VIII is selected from among palladium and platinum, taken by itself or in a mixture.

7. Process according to claim 1, in which said catalyst comprises a content of metal(s) of group VIII of between 0.01 and 4% by weight relative to the total mass of the catalyst.

8. Process according to claim 1, in which said catalyst also comprises at least one metal that is selected from among the metals of groups IIIA, IVA and VIIB, taken by itself or in a mixture.

9. Process according to claim 7, in which said metals of groups IIIA, IVA and VIIB are selected from among gallium, indium, tin and rhenium, taken by itself or in a mixture.

10. Process according to claim 8, in which the metal content of groups IIIA, IVA and VIIB is between 0.01 and 2% by weight relative to the total mass of the catalyst.

11. Process according to claim 1, in which said aromatic fraction comprises either a mixture of only xylenes or only ethylbenzene, or a mixture of xylene(s) and ethylbenzene.

12. Process according to claim 1, in which said process is performed at a temperature of between 300° C. and 500° C., at a partial hydrogen pressure of between 0.3 and 1.5 MPa, at a total pressure of between 0.45 and 1.9 MPa, and at a feed volumetric flow rate, expressed in kilogram of feedstock that is introduced per kilogram of catalyst and per hour, that is between 0.25 and 30 ⁻¹.
